(19) United States
(12) Patent Application Publication
(10) Pub. No.: US 2013/0303899 A1
(43) Pub. Date: Nov. 14, 2013

(54) QUANTITATIVE EVALUATION OF FRACTIONAL REGIONAL VENTILATION USING FOUR-DIMENSIONAL COMPUTED TOMOGRAPHY

(71) Applicants: Nilesh Mistry, Silver Spring, MD (US); Warren D'Souza, Timonium, MD (US); Tejan Diwanji, Germantown, MD (US); Steven Feigenberg, Towson, MD (US)

(72) Inventors: Nilesh Mistry, Silver Spring, MD (US); Warren D'Souza, Timonium, MD (US); Tejan Diwanji, Germantown, MD (US); Steven Feigenberg, Towson, MD (US)

(73) Assignee: UNIVERSITY OF MARYLAND, BALTIMORE, Baltimore, MD (US)

(21) Appl. No.: 13/894,314

(22) Filed: May 14, 2013

Related U.S. Application Data
(60) Provisional application No. 61/646,554, filed on May 14, 2012.

(51) Int. Cl. A61B 6/00

(52) U.S. Cl. CPC A61B 6/5205
USPC 600/425

(57) ABSTRACT

Methods and systems for determining fractional regional ventilation are disclosed. A method includes obtaining first and second lung image data indicative of a first phase and a second phase of a respiratory cycle, respectively, determining an apparent mass ratio k based on the first lung image data and the second lung image data, determining first and second spatially matched lung image data, each including N voxels, based on the first lung image data and the second lung image data, and determining at least one fractional regional ventilation value (FRV value), in accordance with a first equation FRV(n)=(k·ρ2_n−ρ1_n)/ρ1_n. The value of n is a voxel index, ρ1_n is indicative of a density of a voxel n of the first spatially matched lung image data, and ρ2_n is indicative of a density of a voxel n of the second spatially matched lung image data.

### CROSS-REFERENCE TO RELATED APPLICATION

This application claims the benefit of U.S. Provisional Patent Application No. 61/646,554, filed May 14, 2012 in the U.S. Patent and Trademark Office, the disclosure of which is incorporated herein by reference in its entirety.

### FIELD OF THE INVENTION

The present invention generally relates to systems and methods for medical imaging. More specifically, the present invention relates to systems and methods for evaluating lung ventilation function using four-dimensional computed tomography.

### BACKGROUND OF THE INVENTION

The primary physiologic function of the lung is to support gas exchange through ventilation (air reaching the alveoli), perfusion (blood reaching the alveoli), and diffusion of gases across the blood-gas interface. Disrupting any of these functions can adversely affect lung function. For example, many diseases, such as lung cancer, asthma, and chronic obstructive pulmonary disease (COPD), can severely impair ventilation.

In certain circumstances, it is therefore desirable to determine regional lung ventilation. One conventional technique for clinically evaluating lung function is the Pulmonary Function Test (PFT), which provides a global overview of ventilatory disruptions. These ventilatory disruptions, however, are not evenly distributed throughout the lung. As a result, PFT alone fails to capture these regional variations.

Several imaging modalities have been utilized to evaluate regional differences in lung function, including Single Photon Emission Computed Tomography (SPECT), Magnetic Resonance Imaging (MRI) with hyperpolarized gases, and Computed Tomography (CT) with radiodense gases. These techniques, however, have limited clinical application because they require the use of exogenous gases.

Other contemporary techniques for evaluating regional lung ventilation are Hounsfield unit (HU)-based methods using 4-dimensional computed tomography (4D-CT) imaging to extract regional ventilation data. These methods, however, fail to account for the change in mass of lung tissue over a respiratory cycle due to the redistribution of blood. As a result, these methods produce data that are typically inconsistent with a patient's underlying physiology as measured using global lung metrics, such as standardized PFT. These methods also generally produce noisy data.

Accordingly, there exists a need for methods and systems for determining regional lung ventilation, which do not require the use of exogenous gases, which are consistent with a patient's underlying physiology as measured using standardized PFT, and which minimize noise in data.

### SUMMARY OF ILLUSTRATIVE EMBODIMENTS OF THE INVENTION

An aspect of embodiments of the present invention is to substantially address the above and other concerns, and provide methods and systems for determining regional lung ventilation, which do not require the use of exogenous gases, which are consistent with a patient's underlying physiology as measured using standardized PFT, and which minimize noise in data.

The foregoing and/or other aspects of the present invention are achieved by providing an illustrative method of determining fractional regional ventilation, including obtaining first lung image data indicative of a first phase of a respiratory cycle, the first lung image data including at least one first voxel, obtaining second lung image data indicative of a second phase of a respiratory cycle, the second lung image data including at least one second voxel, determining an apparent mass ratio k based on the first lung image data and the second lung image data, determining first spatially matched lung image data including N voxels and second spatially matched lung image data including N voxels, based on the first lung image data and the second lung image data, and determining at least one fractional regional ventilation value (FRV value), in accordance with a first equation FRV(n)=(k·ρ2_n−ρ1_n)/ρ1_n. The value of n is a voxel index greater than or equal to 1 and less than or equal to N. The value of ρ1_n is indicative of a density of a voxel n of the first spatially matched lung image data. The value of ρ2_n is indicative of a density of a voxel n of the second spatially matched lung image data.

The foregoing and/or other aspects of the present invention are also achieved by providing an illustrative system for determining fractional regional ventilation, including a computing system programmed with image analysis software. The computing system is adapted to obtain first lung image data indicative of a first phase of a respiratory cycle, the first lung image data including at least one first voxel. The computing system is further adapted to obtain second lung image data indicative of a second phase of a respiratory cycle, the second lung image data including at least one second voxel. The computing system is further adapted to determine an apparent mass ratio k based on the first lung image data and the second lung image data. The computing system is further adapted to determine first spatially matched lung image data including N voxels and second spatially matched lung image data including N voxels, based on the first lung image data and the second lung image data. The computing system is further adapted to determine at least one fractional regional ventilation value (FRV value), in accordance with a first equation FRV(n)=(k·ρ2_n−ρ1_n)/ρ1_n. The value of n is a voxel index greater than or equal to 1 and less than or equal to N. The value of ρ1_n is indicative of a density of a voxel n of the first spatially matched lung image data. The value of ρ2_n is indicative of a density of a voxel n of the second spatially matched lung image data.

The foregoing and/or other aspects of the present invention are also achieved by providing an illustrative system for determining fractional regional ventilation, including a computing system programmed with image analysis software. The computing system is adapted to obtain first image data and second image data from a scanning system. The computing system is adapted to determine first lung image data indicative of a first phase of a respiratory cycle from the first image data, the first lung image data including at least one first voxel. The computing system is further adapted to determine second lung image data indicative of a second phase of a respiratory cycle from the second image data, the second lung image data including at least one second voxel. The computing system is further adapted to determine an apparent mass ratio k based on the first lung image data and the second lung image data. The computing system is further adapted to determine first spatially matched lung image data including N voxels and second spatially matched lung image data including N voxels, based on the first lung image data and the second lung image data. The computing system is further adapted to determine at least one fractional regional ventilation value (FRV value), in accordance with a first equation FRV(n)=(k·ρ2_n−ρ1_n)/ρ1_n. The value of n is a voxel index greater than or equal to 1 and less than or equal to N. The value of ρ1_n is indicative of a density of a voxel n of the first spatially matched lung image data. The value of ρ2_n is indicative of a density of a voxel n of the second spatially matched lung image data.

Additional and/or other aspects and advantages of the present invention will be set forth in the description that follows, or will be apparent from the description, or may be learned by practice of the invention.

### BRIEF DESCRIPTION OF THE DRAWINGS

The various objects, advantages and novel features of illustrative embodiments of the present invention will be more readily appreciated from the following detailed description when read in conjunction with the appended drawings, in which:

FIG. 1 shows a fractional regional ventilation map in accordance with an illustrative embodiment of the present invention;

FIG. 2 shows a histogram of fractional regional ventilation values in accordance with an illustrative embodiment of the present invention;

FIG. 3 shows a scatter plot for ten phases of the respiratory cycles of seven subjects, in accordance with an illustrative embodiment of the present invention.

FIG. 4 shows a graph comparing tidal volume estimated using an ABC system with that calculated from FRV for the seven patients in accordance with an illustrative embodiment of the present invention.

FIG. 5 shows FRV values extracted using FB, AV, and ABC breathing maneuvers in an axial slice and a coronal projection created from the three-dimensional volume for a representative patient, in accordance with an illustrative embodiment of the present invention;

FIG. 6 shows a mean tidal volume (V_{T}) value for five patients from three different breathing maneuvers, in accordance with an illustrative embodiment of the present invention.

FIGS. 7A-F show lung ventilation maps generated using FRV methods in accordance with an illustrative embodiment of the present invention, and lung ventilation maps using conventional HU-based methods;

FIG. 8 shows a method of determining fractional regional ventilation in accordance with an illustrative embodiment of the present invention;

FIG. 9A shows lung image data at nine phases of a respiratory cycle, in accordance with an illustrative embodiment of the present invention;

FIG. 9B shows a plot of a measured volume of the lung with respect to phases of respiratory cycle, in accordance with an illustrative embodiment of the present invention; and

FIG. 10 shows a combination of a system for determining fractional regional ventilation and a scanning system, in accordance with an illustrative embodiment of the present invention.

### DETAILED DESCRIPTION OF THE ILLUSTRATIVE EMBODIMENTS

As will be appreciated by one skilled in the art, there are numerous ways of carrying out the examples, improvements, and arrangements of a system for determining regional lung ventilation in accordance with embodiments of the present invention disclosed herein. Although reference will be made to the illustrative embodiments depicted in the drawings and the following descriptions, the embodiments disclosed herein are not meant to be exhaustive of the various alternative designs and embodiments that are encompassed by the disclosed invention, and those skilled in the art will readily appreciate that various modifications may be made without departing from scope of the invention.

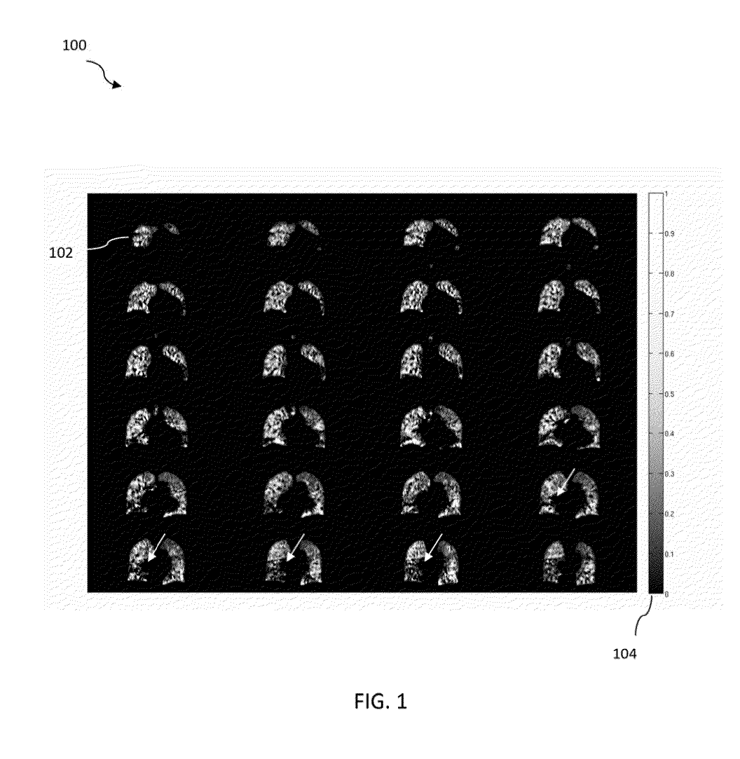

Illustrative embodiments in accordance with the present invention are depicted in FIGS. 1-4. In some illustrative embodiments in accordance with the present invention, methods and systems for determining fractional regional ventilation using 4-dimensional computed tomography (4D-CT) imaging account for tissue-based mass changes over a respiratory cycle. In some illustrative embodiments in accordance with the present invention, these methods and systems do not require the use of exogenous gases, can produce data consistent with a patient's underlying physiology as measured using global lung metrics, such as standardized PFT, and can minimize noise in data.

In some illustrative embodiments in accordance with the present invention, algorithms are implemented by a computing system programmed with image analysis software, including, but not limited to, the open source segmentation and registration toolkits (ITK) (currently available on http://www.itk.org), Matlab® (by Mathworks®), C, C++, IDL and ImageJ® (currently available on httpl/rsbweb.nih.gov/ij/).

Determination of Fractional Regional Ventilation (FRV):

Generally:

In an illustrative embodiment in accordance with the present invention, CT image data expressed in HU are indicative of density (ρ, g/cm3), and are defined by the equation ρ=1+(HU/1000). Using the relationship m=ρ·V between mass (m), volume (V), and density (ρ), one can define the mass of a given volume element or voxel (VOX) as the product of the density and volume of its components. The two main components of the pulmonary system are air and tissue; hence the density of any voxel is expressed as

$${ρ}_{VOX} = \frac{( {ρ}_{AIR} {V}_{AIR} + {ρ}_{TISSUE} {V}_{TISSUE} )}{{V}_{VOX}} , \qquad ( Equation 1 )$$

where ρ_{VOX}, ρ_{AIR}, and ρ_{TISSUE} are indicative of the density of the voxel, the density of air in the voxel, and the density of tissue in the voxel, respectively, and where V_{VOX}, V_{AIR}, and V_{TISSUE} are indicative of the volume of the voxel, the volume of air in the voxel, and the volume of tissue in the voxel, respectively.

Because the density of humid air in the lung is typically, though not necessarily, about 0.001 g/cm³ and that of lung tissue is typically, though not necessarily, about 1.1 g/cm³, in an illustrative embodiment in accordance with the present invention it is assumed that changes in mass are driven by changes in the tissue component, and changes in volume are primarily driven by the air component in that voxel.

In an illustrative embodiment in accordance with the present invention, fractional regional ventilation (FRV) is defined for each voxel using the equation FRV=(V_{IN}−V_{EX})/V_{EX}, where V_{IN} is indicative of the volume of lung (i.e., air and tissue) in a voxel at an inhale phase of a respiratory cycle and V_{EX} is indicative of the volume of lung (i.e., air and tissue) in a voxel at an exhale phase of a respiratory cycle in a given voxel. Summing the numerator (V_{IN}−V_{EX}) for all the voxels in the lung yields a tidal volume (V_{T}). Furthermore, substituting volume with mass and density yields

$$FRV = \frac{\frac{{m}_{IN}}{{ρ}_{IN}} - \frac{{m}_{EX}}{{ρ}_{EX}}}{\frac{{m}_{EX}}{{ρ}_{EX}}} , \qquad ( Equation 2 )$$

where m_{IN} and m_{EX} are indicative of the mass of lung (i.e., air and tissue) in a voxel at an inhale phase of a respiratory cycle and that at an exhale phase of a respiratory cycle, respectively, and ρ_{IN} and ρ_{EX} are indicative of the density of lung (i.e., air and tissue) in a voxel at an inhale phase of a respiratory cycle and that at an exhale phase of a respiratory cycle, respectively. Further simplification of Equation 2 yields

$$FRV = \frac{{m}_{IN} {ρ}_{EX} - {m}_{EX} {ρ}_{IN}}{{m}_{EX} {ρ}_{IN}} . \qquad ( Equation 3 )$$

“Apparent Mass”:

In an illustrative embodiment in accordance with the present invention, each voxel has a fixed volume governed by the resolution of a scanning system adapted to provide information related to density, including, but not limited to, a computed tomography (CT) system. The image data indicates a composite density map of its underlying components. Therefore, one determines an “apparent mass” value as a product of individual voxel density and individual voxel volume. Changes in this “apparent mass” value are indicative of cyclical changes in blood distribution within the pulmonary parenchyma, corresponding to the respiratory cycle. These changes can be due to the distention of blood vessels and respiration induced variations in cardiac output. By correcting the densities in CT image data for “apparent mass”, one can account for changes in the blood volume through the respiratory cycle. For example, a ratio of “apparent mass” between an inhale phase and an exhale phase is obtained as a relationship between an inhale mass and an exhale mass. An “apparent mass” ratio is, thus, expressed as

$$k = \frac{{m}_{IN}}{{m}_{EX}} . \qquad ( Equation 4 )$$

Equation 3 is, thus, rewritten as

$$FRV = \frac{k {ρ}_{EX} - {ρ}_{IN}}{{ρ}_{IN}} , \qquad ( Equation 5 )$$

which provides an expression for mass-corrected FRV.

In an illustrative embodiment in accordance with the present invention, the “apparent mass” ratio k is determined for the entire lung at a particular phase of respiration, by approximating the ratio

$$\frac{{m}_{IN}}{{m}_{EX}}$$

in Equation 4 using mₚₕ=Σ_{lung@ph}ρᵥₒₓνᵥₒₓ (Equation 6), where ph is a phase of a respiratory cycle, and mp is indicative of the mass of lung at phase ph. The “apparent mass” ratio k is, thus, treated as a constant over the entire lung, such that

$$k = \frac{{Σ}_{lung @ IN} {ρ}_{vox} {v}_{vox}}{{Σ}_{lung @ EX} {ρ}_{vox} {v}_{vox}} . \qquad ( Equation 7 )$$

In an illustrative embodiment in accordance with the present invention, FRV is determined in accordance with Equation 5 on a voxel-by-voxel basis by spatially matching image data indicative of an inhale phase of respiration with image data indicative of an exhale phase of respiration. The resulting image data contain an identical number of voxels. For example, matching can be accomplished using any deformable image registration algorithm known in the art, including, but not limited to, a “Demons” deformable image registration algorithm with a multi-resolution scheme, or using any b-splines method known in the art, including, but not limited to, using Image Registration Toolkit (IRTK). Voxel-size can be determined using a native CT-reconstruction spatial resolution.

In an illustrative embodiment in accordance with the present invention, an algorithm is implemented using a computing system programmed with image analysis software, including, but not limited to, the open source segmentation and registration toolkits (ITK) (currently available on http://www.itk.org), Matlab®) (by Mathworks®), C, C++, IDL and ImageJ® (currently available on httpJ/rsbweb.nih.gov/ij/).

In an illustrative embodiment in accordance with the present invention, mass-corrected FRV values for each voxel are estimated using Equation 5 and a constant k, for each of the spatially matched image data.

Breathing Manoeuvers:

In an illustrative embodiment in accordance with the present invention, a patient breathes during a scan according to any breathing manoeuvers known in the art, including, but not limited to, free breathing (FB), breathing with audio-visual guidance (AV), and breathing with active breathing control (ABC). FB is performed without respiratory coaching, while AV and ABC require respiratory coaching.

In an illustrative embodiment in accordance with the present invention, breathing with AV is captured using a real-time position management (RPM) system, including, but not limited to, the one produced by Varian Medical Systems, Palo Alto, Calif. The RPM system captures the motion of an infrared block that is placed on the patient's abdomen as a surrogate for respiration. A patient is shown this motion pattern as biofeedback, and is instructed to follow audio-visual instructions, such as “breathe in”/“breathe out” instructions. Imaging is conducted while the patient follows these audio-visual cues. The breathing pattern captured by the RPM system during imaging is also used to assist image reconstruction at different phases of respiration.

In an illustrative embodiment in accordance with the present invention, breathing with ABC is acquired while a patient is coached using an active breathing control system, including, but not limited to, the Active Breathing Coordinator™ (ABC) system. The patient's breathing pattern is acquired using a mouthpiece that monitors air-flow. These breathing traces, along with audio instructions, are employed as feedback for the patient during the scan. In an illustrative embodiment in accordance with the present invention, the ABC system uses a breathing controller to enable a specified volume of air to be delivered to the patient via the mouthpiece. Alternatively, the ABC system is used in a “passive mode”, i.e., the mouthpiece simply monitors the volume of air inhaled during each breath. This data is averaged to infer tidal volume (V_{T}) during the imaging session. All scans are reconstructed at a number of phases (e.g., 10 phases) over the entire respiratory cycle.

Representation of Fractional Regional Ventilation (FRV):

In an illustrative embodiment in accordance with the present invention, FRV is be represented as a fractional regional ventilation map (FRV map). Alternatively, the distribution of FRV values is represented in a histogram.

FIG. 1 shows an FRV map 100 in accordance with an illustrative embodiment of the present invention. Specifically, the FRV map 100 shows 24 coronal slices 102, covering the lung, of anatomical CT images at full inhalation overlaid with FRV from a representative dataset. Images are displayed using a scale ranging between 0-1.0 and the corresponding color-map 104 ranging from black to white. In other illustrative embodiments in accordance with the present invention, any number of coronal slices are represented using any color-map range. The lower values represent a smaller fractional change in ventilation, and the higher values represent a larger fractional change in ventilation. Regions surrounding a tumor (indicated on some coronal slices by arrows) show an area of reduced ventilation. Areas of reduced ventilation are also visible in the upper left lung. This type of representation can be useful in assessing the health of the lung (i.e., as indicated by lung ventilation), for example, in cases of Chronic Obstructive Pulmonary Disease (COPD), asthma, or emphysema. This type of representation can also indicate the evaluation of lung injury, prior to radiation therapy (for treatment planning), or prior to a surgical intervention to determine which parts of the lung can be surgically removed without compromising lung function. This information can also be useful in follow-up assessments of lung function during and post-treatment.

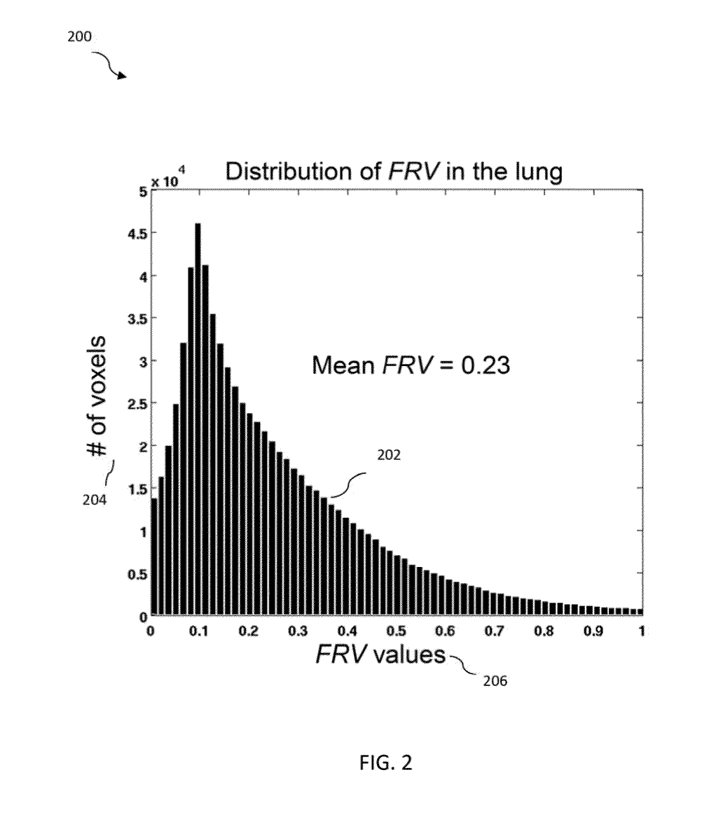

FIG. 2 shows a histogram 200 of FRV values in accordance with an illustrative embodiment of the present invention. The histogram 200 shows a distribution of FRV values expressed as a bar graph 202 representing the number of voxels 204 associated with various FRV values 206 for one patient. This type of representation can be useful in relating regional lung function to global lung function as evaluated, for example, using spirometry. Such information can be used to simultaneously derive a global ratio of tidal volume to end-expiratory-lung-volume, indicative of how well a patient is breathing. A typical problematic distribution may result in mean values dose to 0 g/mL (for example, about 0.05-0.10), indicating that the person is only able to take shallow breaths. Preferably, a patient is coached to breathe deeply during tests producing these data. In a study of seven subjects, the mean of the distribution of FRV (fractional regional ventilation) for all seven subjects estimated from 4D-CT images acquired during ABC was 0.22±0.07. A majority of voxels in the lung show a volume change in the order of 15-30% of end-expiratory total lung volume, with the mean ˜0.23.

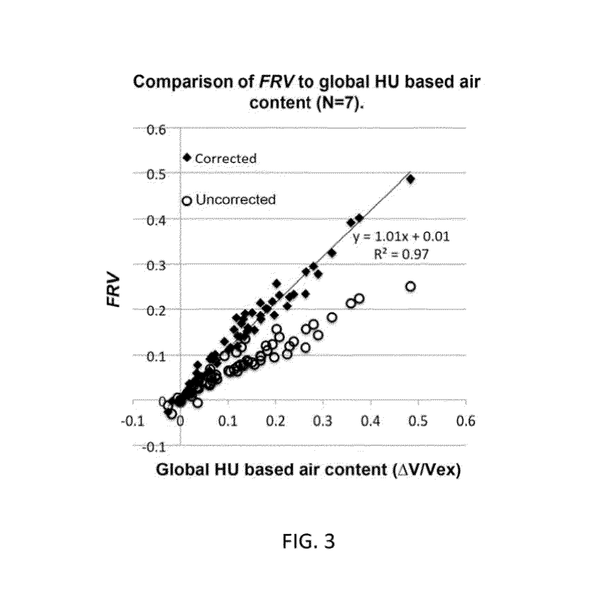

FIG. 3 shows a scatter plot for ten phases of the respiratory cycles of seven subjects, in accordance with an illustrative embodiment of the present invention. The comparison between the global and regional changes in ventilation shows a strong linear correlation (slope of 1.01, R2 of 0.97) between the measured global air content based assessment of global ventilation and regional ventilation calculated using the “mass corrected” FRV. FIG. 3 also illustrates that this correlation does not hold when uncorrected FRV is used.

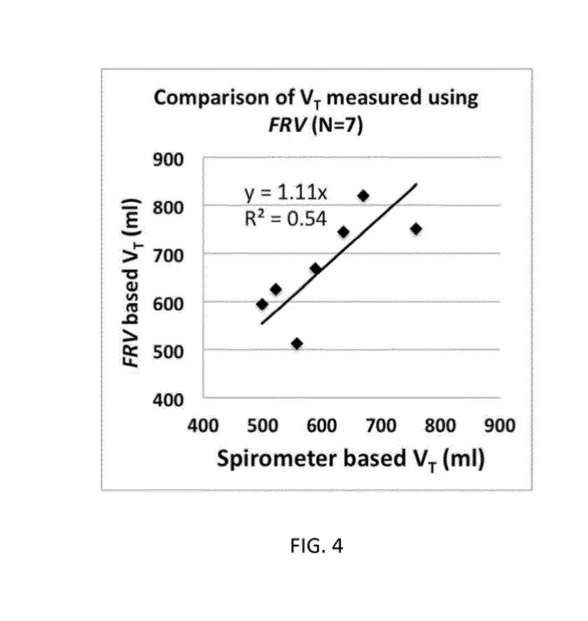

FIG. 4 shows a graph comparing tidal volume estimated using an ABC system with that calculated from FRV for the seven patients in accordance with an illustrative embodiment of the present invention. The relationship between the FRV-based estimates of V_{T} was found to be linear (slope 1.11), indicating a high accuracy of measurements made using methods in accordance with an illustrative embodiment of the present invention.

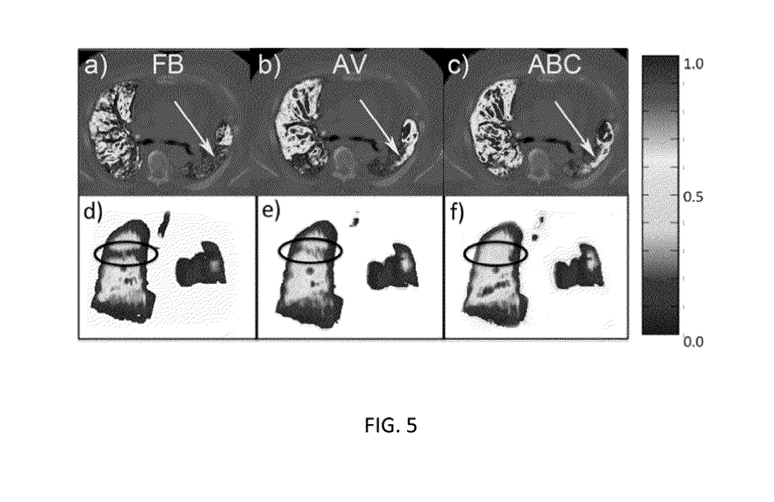

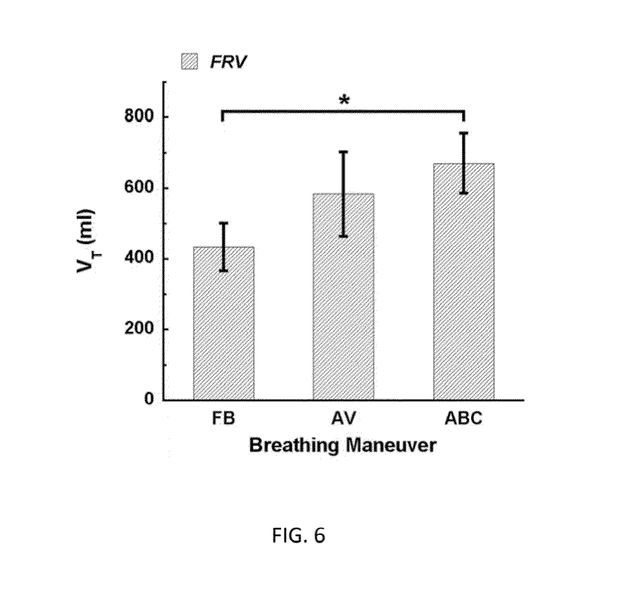

FIG. 5 shows FRV values extracted using FB, AV, and ABC breathing maneuvers in an axial slice (a, b, c), and a coronal projection (d, e, f) created from the three-dimensional volume for a representative patient, in accordance with an illustrative embodiment of the present invention. Arrows indicate areas of increased FRV as seen in FIGS. 5a-c. Similar changes are also visible in the coronal projections. The ovals shown in d-f (FB, AV, and ABC, respectively) indicate an area in the right upper lobe that shows additional recruitment due to changing breathing patterns. These regional changes are also reflected in the global measures of tidal volume measured by FRV, in accordance with an illustrative embodiment of the present invention, as shown in FIG. 6. Higher tidal volumes are consistently seen in a group of five subjects using the ABC biofeedback coaching as opposed to FB or AV.

FIG. 6 shows a mean tidal volume (V_{T}) value for five patients from three different breathing maneuvers in accordance with an illustrative embodiment of the present invention. FIG. 6 shows a much larger tidal volume when the patients breathe using ABC (where * indicates p<0.01).

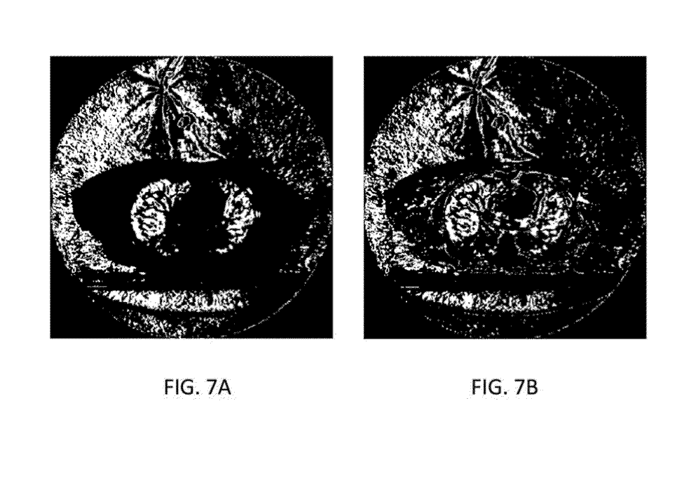

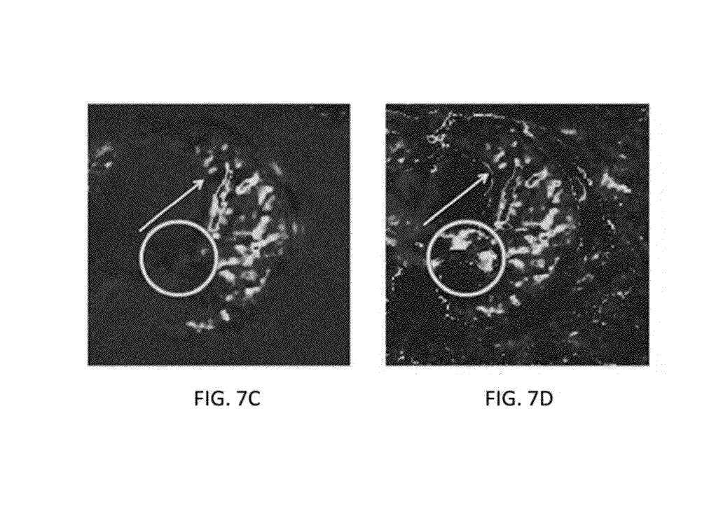

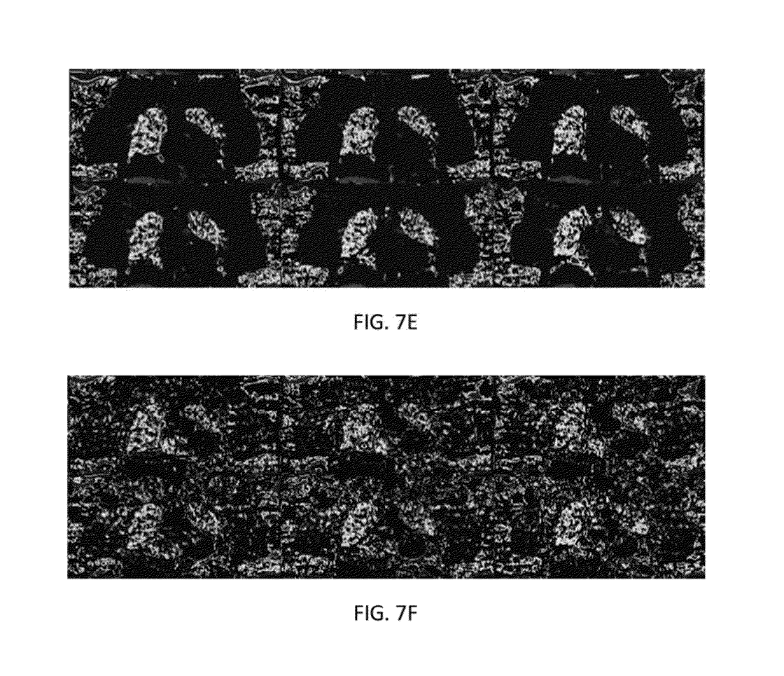

FIGS. 7A-F show lung ventilation maps (FIGS. 7A, 7C and 7E) generated using FRV methods in accordance with an illustrative embodiment of the present invention, and lung ventilation maps (FIGS. 7B, 7D and 7F) using conventional HU-based methods. FIGS. 7A, 7C and 7E appear less noisy than FIGS. 7B, 7D and 7F, thereby highlighting an advantage of illustrative methods in accordance with some illustrative embodiments of the present invention, over methods that do not correct for the mass of the entire lung over a respiratory cycle.

### EXAMPLES

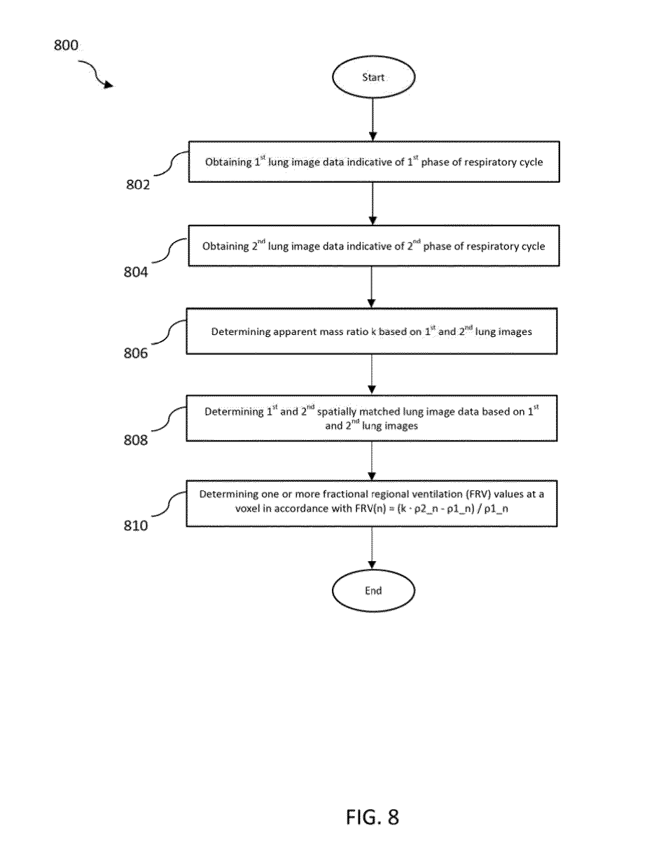

FIG. 8 shows a method 800 of determining fractional regional ventilation in accordance with an illustrative embodiment of the present invention. The method 800 includes obtaining first lung image data indicative of a first phase of a respiratory cycle, the first lung image data including at least one first voxel, as step 802, obtaining second lung image data indicative of a second phase of a respiratory cycle, the second lung image data including at least one second voxel, at step 804, determining an apparent mass ratio k based on the first lung image data and the second lung image data at step 806, determining first spatially matched lung image data including N voxels and second spatially matched lung image data including N voxels, based on the first lung image data and the second lung image data, at step 808, and determining at least one fractional regional ventilation value (FRV value), in accordance with a first equation FRV(n)=(k·ρ2_n−ρ1_n)/ρ1_n at step 810. The value of n is a voxel index greater than or equal to 1 and less than or equal to N. The value of ρ1_n is indicative of a density of a voxel n of the first spatially matched lung image data. The value of ρ2_n is indicative of a density of a voxel n of the second spatially matched lung image data.

In an illustrative embodiment in accordance with the present invention, values of p are determined using values of voxels of an image. For example, in an image produced using a CT system, a ρ value corresponds to an HU value of the image at a particular voxel.

In an illustrative embodiment in accordance with the present invention, obtaining the first lung image data includes determining the first lung image data from first image data indicative of the first phase of the respiratory cycle.

In an illustrative embodiment in accordance with the present invention, obtaining the second lung image data includes determining the second lung image data from second image data indicative of the second phase of the respiratory cycle.

In an illustrative embodiment in accordance with the present invention, image data indicative of a phase of a respiratory cycle is obtained by scanning a patient at the phase of a respiratory cycle. For example, scanning is performed using a scanning system adapted to provide information related to density, including, but not limited to, a computed tomography (CT) system. Alternatively, in an illustrative embodiment in accordance with the present invention, image data indicative of a phase of a respiratory cycle are obtained from an external device, by wired or wireless communication, or by removable media.

In an illustrative embodiment in accordance with the present invention, lung image data are determined using any shape recognition method known in the art, including, but not limited to, at least one of thresholding, image morphological operations, voxel connectivity, or manual segmentation. For example, thresholding is performed by selecting from an image voxels with HU values within a desired range. In an illustrative embodiment in accordance with the present invention, image morphological operations include, but are not limited to, the use of a process of binary opening that aims to eliminate regions outside the lung from the image. Alternatively, in an illustrative embodiment in accordance with the present invention, lung image data indicative of a phase of a respiratory cycle are obtained from an external device, by wired or wireless communication, or by removable media.

In an illustrative embodiment in accordance with the present invention, the first lung image data and the second lung image data are selected from a plurality of lung image data, each lung image data indicative of a lung at one of a plurality of phases of a respiratory cycle. In an illustrative embodiment in accordance with the present invention, the plurality of lung image data are binned using any binning method known in the art, including, but not limited to, time-based binning or amplitude-based binning.

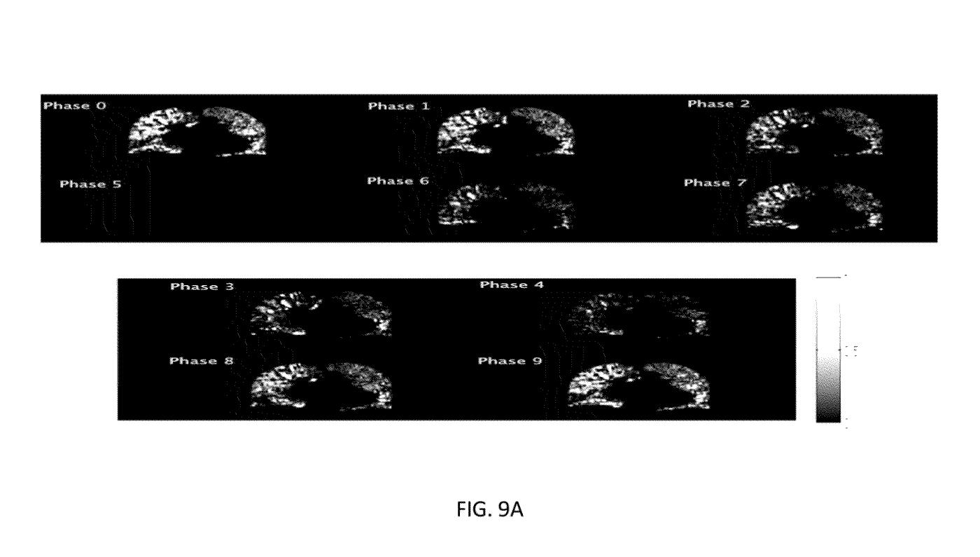

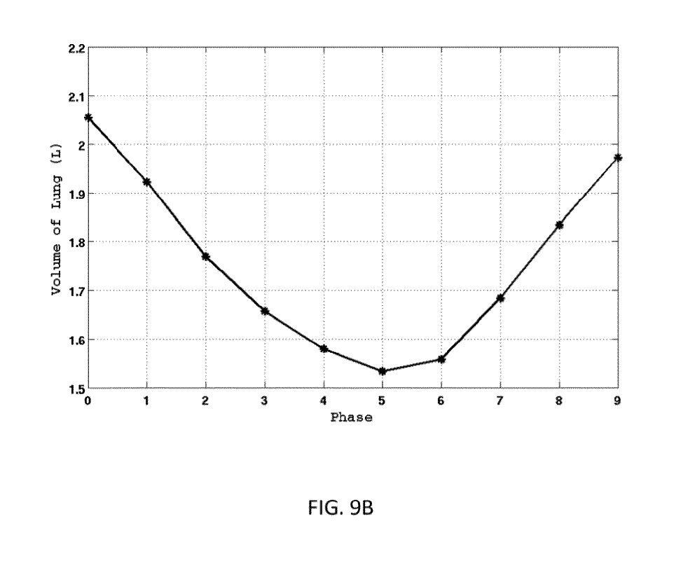

In an illustrative embodiment in accordance with the present invention, the first phase is an inhale phase of the respiratory cycle. Typically, the lung is largest in volume at an inhale phase of a respiratory cycle. Accordingly, in an illustrative embodiment in accordance with the present invention, the first lung image data selected from the plurality of lung image data by selecting lung image data with the greatest number of voxels from the plurality of lung image data. Typically, in a sequence Phase 0 . . . Phase 9 of image lung data (as illustrated in FIGS. 9A-B), Phase 0 is indicative of an inhale phase of the respiratory cycle. Therefore, alternatively, in an illustrative embodiment in accordance with the present invention, Phase 0 from a sequence Phase 0 . . . Phase 9 of image lung data are selected as the first lung image data without counting voxels.

Alternatively, in an illustrative embodiment in accordance with the present invention, the first lung image data are selected by looping through all lung image data from the plurality of lung image data, to produce a sequence of first image lung data, and to yield a sequence of FRV values. This can generate an animation showing the progression of ventilation over time.

In an illustrative embodiment in accordance with the present invention, the second phase is an exhale phase of the respiratory cycle. Typically, the lung is least at an exhale phase of a respiratory cycle. Accordingly, in an illustrative embodiment in accordance with the present invention, the second lung image data are selected from the plurality of lung image data by selecting lung image data with the least number of voxels from the plurality of lung image data. Typically, in a sequence Phase 0 . . . Phase 9 of image lung data, Phase 4, Phase 5 or Phase 6 is indicative of an exhale phase of the respiratory cycle (as illustrated in FIGS. 9A-B).

In an illustrative embodiment in accordance with the present invention, k is determined at step 806 in accordance with an equation k=m1/m2. The value of m1 is indicative of a mass of lung at the first phase, and the value of m2 is indicative of a mass of lung at the second phase.

In an illustrative embodiment in accordance with the present invention, m_{—}1 is determined in accordance with an equation m1=Σ(over 1^{st} voxels at 1^{st} phase)[ρ1_vox·v1_vox]. The value of vox is a voxel index. The value of m1 is indicative of a sum of the products of the density ρ1_vox and the volume v1_vox of each of the first voxels. In an illustrative embodiment in accordance with the present invention, values of v1_vox are constant values corresponding to the volume of each voxel of the first image data, according to the resolution of the scanning system. Similarly, m2 is determined in accordance with an equation m2=Σ(over 2^{nd} voxels at 2^{nd} phase)[ρ_{—}2_vox·v_{—}2_vox]. The value of vox is a voxel index. The value of m2 is indicative of a sum of the products of the density ρ2_vox and the value v2_vox of each of the second voxels.

In an illustrative embodiment in accordance with the present invention, the method further includes storing the value of k, for example, on computer-readable media. By storing the value of k, k need not be computed again. Specifically, in an illustrative embodiment in accordance with the present invention, the stored value of k is used in the determination of the FRV values at any voxel, in accordance with FRV(n)=(k·ρ2_n−ρ1_n)/ρ1_n, at step 810.

In an illustrative embodiment in accordance with the present invention, the first spatially matched lung image data and the second spatially matched lung image data are determined at step 808 by spatially matching the first lung image data and the second lung image data. As a result of spatial matching, first spatially matched lung image data and second spatially matched lung image data each include N voxels, and each voxel of the first spatially matched lung image data corresponds to one voxel in the second spatially matched lung image data.

In an illustrative embodiment in accordance with the present invention, spatially matching is accomplished using any deformable image registration algorithm known in the art, including, but not limited to, a “Demons” deformable image registration algorithm with a multi-resolution scheme, or using any b-splines methods known in the art, including, but not limited to, using Image Registration Toolkit (IRTK). Voxel-size is determined using a native CT-reconstruction spatial resolution.

In an illustrative embodiment in accordance with the present invention, an algorithm is implemented using a computing system programmed with image analysis software, including, but not limited to, the open source segmentation and registration toolkits (ITK) (currently available on http://www.itk.org), Matlab® (by Mathworks®), C, C++, IDL and ImageJ® (currently available on http://rsbweb.nih.gov/ij/).

In an illustrative embodiment in accordance with the present invention, a computing system programmed with image analysis software is adapted to perform substantially all steps of method 800.

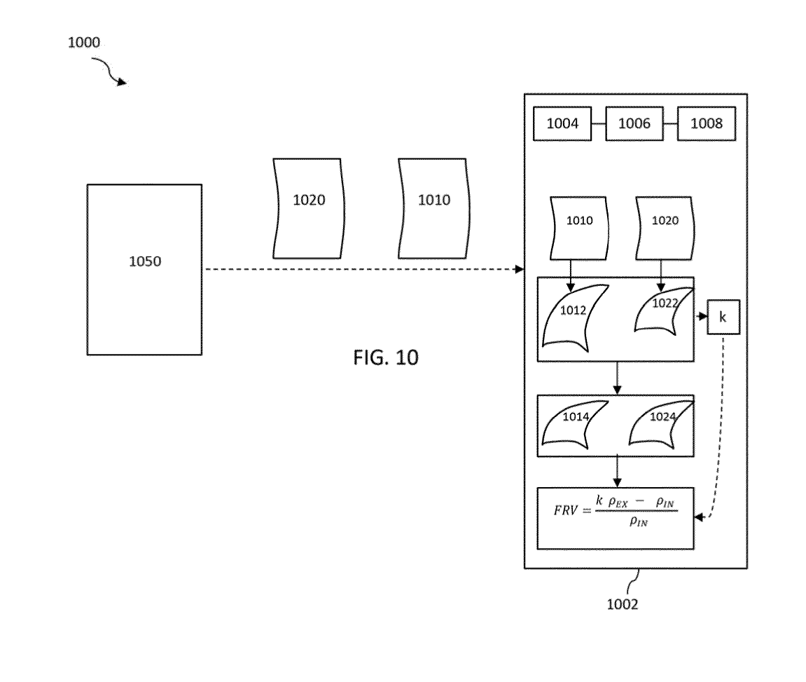

FIG. 10 shows a combination of a system 1000 for determining fractional regional ventilation and a scanning system 1050, in accordance with an illustrative embodiment of the present invention. The system for determining fractional regional ventilation 1000 includes a computing system 1002 programmed with image analysis software. In an illustrative embodiment in accordance with the present invention, the computing system 1002 includes computer-readable media 1004, a processing module 1006 and a communication module 1008, all electrically or communicatively coupled. In an illustrative embodiment in accordance with the present invention, the communication module 1008 is adapted for wired or wireless communication, including, but not limited to, USB, serial, LAN, WLAN, cell communication, Bluetooth®, ZigBee®, infrared and other RF communication methods known in the art. In an illustrative embodiment in accordance with the present invention, the computing system 1002 is adapted for data input or output by removable media.

In an illustrative embodiment in accordance with the present invention, the computing system 1002 is adapted to obtain first image data 1010 and second image data 1020 from a scanning system 1050. The computing system 1002 is further adapted to determine first lung image data 1012 from the first image data 1010, first lung image data 1012 including one or more first voxeis, and to determine second lung image data 1022 from the second image data 1020, second lung image data 1022 including one or more second voxels. The first lung image data 1012 includes one or more first voxels and is indicative of a first phase of a respiratory cycle. The second lung image data 1022 includes one or more second voxels and is indicative of a second phase of a respiratory cycle. The computing system 1002 is further adapted to determine an apparent mass ratio k based on the first lung image data 1012 and the second lung image data 1022. The computing system 1002 is further adapted to determine first spatially matched lung image data 1014 including N voxels and second spatially matched lung image data 1024 including N voxels, based on the first lung image data 1012 and the second lung image data 1022. The computing system 1002 is further adapted to determine at least one fractional regional ventilation value (FRV value), in accordance with a first equation FRV(n)=(k·ρ2_n−ρ1_n)/ρ1_n. The value of n is a voxel index greater than or equal to 1 and less than or equal to N. The value of ρ1_n is indicative of a density of a voxel n of the first spatially matched lung image data 1014. The value of ρ2_n is indicative of a density of a voxel n of the second spatially matched lung image data 1024.

In an illustrative embodiment in accordance with the present invention, the computing system 1002 is adapted to obtain a first image data 1010 and a second image data 1020 from a scanning system 1050 by one of wired communication, wireless communication, or removable computer-readable media.

In an illustrative embodiment in accordance with the present invention, the scanning system 1050 includes any scanning system adapted to provide information related to density, including, but not limited to, a computed tomography (CT) system. Alternatively, in an illustrative embodiment in accordance with the present invention, image data indicative of a phase of a respiratory cycle are obtained from an external device, by wired or wireless communication, or by removable media.

In an illustrative embodiment in accordance with the present invention, values of ρ are determined using values of voxels of an image. For example, in an image produced using the scanning system 1050, a ρ value corresponds to an HU value of the image at a particular voxel.

In an illustrative embodiment in accordance with the present invention, the computing system 1002 is adapted to determine lung image data using any shape recognition method known in the art, including, but not limited to, at least one of thresholding, image morphological operations, voxel connectivity, or manual segmentation. For example, thresholding is performed by selecting from an image voxels with HU values within a desired range. In an illustrative embodiment in accordance with the present invention, image morphological operations include, but are not limited to, the use of a process of binary opening that aims to eliminate regions outside the lung from the image. Alternatively, in an illustrative embodiment in accordance with the present invention, lung image data indicative of a phase of a respiratory cycle are obtained from an external device, by wired or wireless communication, or by removable media.

In an illustrative embodiment in accordance with the present invention, the computing system 1002 is adapted to select the first lung image data and the second lung image data from a plurality of lung image data, each lung image data indicative of a lung at one of a plurality of phases of a respiratory cycle. In an illustrative embodiment in accordance with the present invention, the plurality of lung image data are binned using any binning method known in the art, including, but not limited to, time-based binning or amplitude-based binning.

In an illustrative embodiment in accordance with the present invention, the first phase is an inhale phase of the respiratory cycle. Typically, the lung is largest in volume at an inhale phase of a respiratory cycle. Accordingly, in an illustrative embodiment in accordance with the present invention, the first lung image data are selected from the plurality of lung image data by selecting lung image data with the greatest number of voxels from the plurality of lung image data. Typically, in a sequence Phase 0 . . . Phase 9 of image lung data (as illustrated in FIGS. 9A-B), Phase 0 is indicative of an inhale phase of the respiratory cycle. Therefore, alternatively, in an illustrative embodiment in accordance with the present invention, Phase 0 from a sequence Phase 0 . . . Phase 9 of image lung data are selected as the first lung image data without counting voxels.

Alternatively, in an illustrative embodiment in accordance with the present invention, the first lung image data are selected by looping through all lung image data from the plurality of lung image data, to produce a sequence of first image lung data, and to yield a sequence of FRV values. This can generate an animation showing the progression of ventilation over time.

In an illustrative embodiment in accordance with the present invention, the second phase is an exhale phase of the respiratory cycle. Typically, the lung is least at an exhale phase of a respiratory cycle. Accordingly, the second lung image data are selected from the plurality of lung image data by selecting lung image data with the least number of voxels from the plurality of lung image data. Typically, in a sequence Phase 0 . . . Phase 9 of image lung data, Phase 4, Phase 5 or Phase 6 is indicative of an exhale phase of the respiratory cycle (as illustrated in FIGS. 9A-B).

In an illustrative embodiment in accordance with the present invention, the computing system 1002 is adapted to determine k in accordance with an equation k=m1/m2. The value of m1 is indicative of a mass of lung at the first phase, and the value of m2 is indicative of a mass of lung at the second phase.

In an illustrative embodiment in accordance with the present invention, the computing system 1002 is adapted to determine m_{—}1 in accordance with an equation m1=Σ(over 1^{st} voxels at 1^{st} phase)[ρ1_vox·v1_vox]. The value of vox is a voxel index. The value of m1 is indicative of a sum of the products of the density ρ1_vox and the volume v1_vox of each of the first voxels. In an illustrative embodiment in accordance with the present invention, values of v1_vox are constant values corresponding to the volume of each voxel of the first image data, according to the resolution of the scanning system. Similarly, in an illustrative embodiment in accordance with the present invention, the computing system 1002 is adapted to determine m2 in accordance with an equation m2=Σ(over 2^{nd} voxels at 2^{nd} phase)[ρ_{—}2_vox−v_{—}2_vox]. The value of vox is a voxel index. The value of m2 is indicative of a sum of the products of the density ρ2_vox and the value v2_vox of each of the second voxels.

In an illustrative embodiment in accordance with the present invention, the computing system 1002 is adapted to store the value of k on computer-readable media 1004. By storing the value of k, k need not be computed again. Specifically, the computing system 1002 is adapted to determine the FRV values at any voxel, in accordance with FRV(n)=(k·ρ2_n−ρ1_n)/ρ1_n and the stored value of k.

In an illustrative embodiment in accordance with the present invention, the computing system 1002 is adapted to determine the first spatially matched lung image data and the second spatially matched lung image data by spatially matching the first lung image data and the second lung image data. As a result of spatial matching, first spatially matched lung image data and second spatially matched lung image data each include N voxels, and each voxel of the first spatially matched lung image data corresponds to one voxel in the second spatially matched lung image data.

In an illustrative embodiment in accordance with the present invention, spatially matching is accomplished using any deformable image registration algorithm known in the art, including, but not limited to, a “Demons” deformable image registration algorithm with a multi-resolution scheme, or using any b-splines methods known in the art, including, but not limited to, using Image Registration Toolkit (IRTK). Voxel-size is determined using a native CT-reconstruction spatial resolution.

In an illustrative embodiment in accordance with the present invention, an algorithm is implemented using a computing system programmed with image analysis software, including, but not limited to, the open source segmentation and registration toolkits (ITK) (currently available on http://www.itk.org), Matlab® (by Mathworks®), C, C++, IDL and ImageJ® (currently available on http://rsbweb.nih.gov/ij/).

The components of the illustrative devices, systems and methods employed in accordance with the illustrated embodiments of the present invention can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers. Examples of the computer-readable recording medium include, but are not limited to, read-only memory (ROM), random-access memory (RAM), CD-ROMs, magnetic tapes, floppy disks, optical data storage devices. It is envisioned that aspects of the present invention can be embodied as carrier waves (such as data transmission through the Internet via wired or wireless transmission paths). A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. The computer-readable recording medium can also be distributed over network-coupled computer systems so that the computer-readable code is stored and executed in a distributed fashion. Also, functional programs, codes, and code segments for accomplishing the present invention can be easily construed as within the scope of the invention by programmers skilled in the art to which the present invention pertains. For example, method steps associated with the illustrative embodiments of the present invention are performed by one or more programmable processors executing a computer program, code or instructions to perform functions (e.g., by operating on input data and/or generating an output). Examples of program instructions include both machine code, such as produced by a compiler, and files containing higher level code that may be executed by the computer using an interpreter. The described hardware devices may be configured to act as one or more software modules in order to perform the operations of the above-described embodiments of the present invention. Method steps can also be performed by, and apparatus of the invention can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

Although only a few illustrative embodiments of the present invention have been described in detail above, those skilled in the art will readily appreciate that many modifications are possible in the illustrative embodiments without materially departing from the novel teachings and advantages of this invention. Accordingly, all such modifications are intended to be included within the scope of this invention.

What is claimed is:

1. A method of determining fractional regional ventilation, comprising:
obtaining first lung image data indicative of a first phase of a respiratory cycle, the first lung image data including at least one first voxel;
obtaining second lung image data indicative of a second phase of a respiratory cycle, the second lung image data including at least one second voxel;
determining an apparent mass ratio (k) based on the first lung image data and the second lung image data;
determining first spatially matched lung image data including N voxels and second spatially matched lung image data including N voxels, based on the first lung image data and the second lung image data; and
determining at least one fractional regional ventilation value (FRV value), in accordance with a first equation FRV(n)=(k·ρ2_n−ρ1_n)/ρ1_n,
wherein n is a voxel index greater than or equal to 1 and less than or equal to N, and
ρ1_n is indicative of a density of a voxel n of the first spatially matched lung image data, and
ρ2_n is indicative of a density of a voxel n of the second spatially matched lung image data.

2. The method of determining fractional regional ventilation of claim 1, wherein the obtaining the first lung image data comprises determining the first lung image data from first image data indicative of the first phase of the respiratory cycle.

3. The method of determining fractional regional ventilation of claim 2, further comprising obtaining the first image data by scanning a patient at the first phase of the respiratory system.

4. The method of determining fractional regional ventilation of claim 2, comprising determining the first lung image data in accordance with at least one of thresholding, image morphological operations, voxel connectivity, and manual segmentation.

5. The method of determining fractional regional ventilation of claim 1, wherein the obtaining the second lung image data comprises determining the second lung image data from second image data indicative of the second phase of the respiratory cycle.

6. The method of determining fractional regional ventilation of claim 5, further comprising obtaining the second image data by scanning a patient at the second phase of the respiratory system.

7. The method of determining fractional regional ventilation of claim 5, comprising determining the second lung image data in accordance with at least one of thresholding, image morphological operations, voxel connectivity, and manual segmentation.

8. The method of determining fractional regional ventilation of claim 1, wherein the first phase is an inhale phase of the respiratory cycle.

9. The method of determining fractional regional ventilation of claim 1, wherein the second phase is an exhale phase of the respiratory cycle.

10. The method of determining fractional regional ventilation of claim 1, further including:
selecting at least one of the first lung image data and the second lung image data from a plurality of lung image data,
wherein each of the plurality of lung image data are indicative of a lung at one of a plurality of phases of a respiratory cycle.

11. The method of determining fractional regional ventilation of claim 10, wherein lung image data having the greatest number of voxels from the plurality of lung image data are selected as the first lung image data.

12. The method of determining fractional regional ventilation of claim 10, wherein lung image data having the least number of voxels from the plurality of lung image data are selected as the second lung image data.

13. The method of determining fractional regional ventilation of claim 1,
wherein k is determined in accordance with a second equation k=m1/m2, and
m1 is indicative of a mass of lung at the first phase, and
m2 is indicative of a mass of lung at the second phase.

14. The method of determining fractional regional ventilation of claim 13, wherein m1 is indicative of a sum of products of a density and a volume of each of the at least one first voxel.

15. The method of determining fractional regional ventilation of claim 13, wherein m2 is indicative of a sum of products of a density and a volume of each of the at least one second voxel.

16. The method of determining fractional regional ventilation of claim 13, further comprising storing k on computer-readable media.

17. The method of determining fractional regional ventilation of claim 1, further comprising determining at least one of the at least one FRV value using a stored value of k.

18. A system for determining fractional regional ventilation, comprising:
a computing system programmed with image analysis software,
wherein the computing system is adapted to obtain first lung image data indicative of a first phase of a respiratory cycle, the first lung image data including at least one first voxel, and
the computing system is further adapted to obtain second lung image data indicative of a second phase of a respiratory cycle, the second lung image data including at least one second voxel, and
the computing system is further adapted to determine an apparent mass ratio (k) based on the first lung image data and the second lung image data, and
the computing system is further adapted to determine first spatially matched lung image data including N voxels and second spatially matched lung image data including N voxels, based on the first lung image data and the second lung image data, and
the computing system is further adapted to determine at least one fractional regional ventilation value (FRV value), in accordance with a first equation FRV(n)=(k·ρ2_n−ρ1_n)/ρ1_n, and
n is a voxel index greater than or equal to 1 and less than or equal to N, and
ρ1_n is indicative of a density of a voxel n of the first spatially matched lung image data, and
ρ2_n is indicative of a density of a voxel n of the second spatially matched lung image data.

19. A system for determining fractional regional ventilation, comprising:
a computing system programmed with image analysis software;
wherein the computing system is adapted to obtain first image data and second image data from a scanning system, and
the computing system is adapted to determine first lung image data indicative of a first phase of a respiratory cycle from the first image data, the first lung image data including at least one first voxel, and
the computing system is further adapted to determine second lung image data indicative of a second phase of a respiratory cycle from the second image data, the second lung image data including at least one second voxel, and
the computing system is further adapted to determine an apparent mass ratio (k) based on the first lung image data and the second lung image data, and
the computing system is further adapted to determine first spatially matched lung image data including N voxels and second spatially matched lung image data including N voxels, based on the first lung image data and the second lung image data, and
the computing system is further adapted to determine at least one fractional regional ventilation value (FRV value), in accordance with a first equation FRV(n)=(k·ρ2_n−ρ1_n)/ρ1_n, and
n is a voxel index greater than or equal to 1 and less than or equal to N, and
ρ1_n is indicative of a density of a voxel n of the first spatially matched lung image data, and
ρ2_n is indicative of a density of a voxel n of the second spatially matched lung image data.

20. A combination of:
the system for determining fractional regional ventilation of claim 19; and
a scanning system,
wherein the computing system programmed with image analysis software is wired or wirelessly communicatively coupled to the scanning system.
